(19) United States
(12) Patent Application Publication
(10) Pub. No.: US 2002/0120329 A1
(43) Pub. Date: Aug. 29, 2002

(54) Moveable intraocular lenses and combinations of intraocular lenses

(75) Inventors: Alan J. Lang, Long Beach, CA (US); Stephen W. Laguette, Laguna Niguel, CA (US)

Correspondence Address: Frank J. Uxa, Stout, Uxa, Buyan & Mullins, LLP, Suite 300, 4 Venture, Irvine, CA, 92618, US

(73) Assignee: Allergan Sales, Inc., Irvine, CA (US)

(21) Appl. No.: 09/795,929

(22) Filed: Feb. 28, 2001

(51) Int. Cl. A61F 2/16

(52) U.S. Cl. 623/6.4; 623/6.44; 623/6.48

(57) ABSTRACT

Intraocular lenses include a reduced size optic adapted to focus light toward a retina of an eye and an at least partially opaque movement assembly coupled to the optic. In one embodiment, the optic has a far vision correction power and the movement assembly is at least partially black and adapted to cooperate with the eye to effect accommodating movement of the optic, preferably upon radial compression by a capsular bag of the eye. The optic preferably vaults anteriorly relative to the movement assembly. Enhanced amounts of accommodation preferably are achieved. Combinations of first and second optics and at least partially opaque movement assembly are also provided.

### BACKGROUND OF THE INVENTION

The present invention is directed to intraocular lenses (IOLs). More particularly, the invention relates to IOLs and combinations of IOLs which are adapted to provide accommodating movement in the eye.

The human eye includes an anterior chamber between the cornea and iris, a posterior chamber, defined by a capsular bag, containing a crystalline lens, a ciliary muscle, a vitreous chamber behind the lens containing the vitreous humor, and a retina at the rear of this chamber. The human eye has a natural accommodation ability. The contraction and relaxation of the ciliary muscle provides the eye with near and distant vision, respectively. This ciliary muscle action shapes the natural crystalline lens to the appropriate optical configuration for focusing light rays entering the eye on the retina.

After the natural crystalline lens is removed, for example, because of cataract or other condition, a conventional, monofocal IOL can be placed in the posterior chamber. Such a conventional IOL has very limited, if any, accommodating ability. However, the wearer of such an IOL continues to require the ability to view both near and far (distant) objects. Corrective spectacles may be employed as a useful solution. Recently, multifocal IOLs without accommodating movement have been used to provide near/far vision correction.

Attempts have been made to provide IOLs with accommodating movement along the optical axis of the eye as an alternative to shape changing. Examples of such attempts are set forth in Levy U.S. Pat. No. 4,409,691 and several patents to Cumming, including U.S. Pat. Nos. 5,674,282 and 5,496,366. The disclosure of each of these patents is incorporated herein by reference. One problem that exists with such IOLs is that they often cannot move sufficiently to obtain the desired accommodation. Certain aspects of this problem have been addressed in commonly assigned U.S. patent applications Ser. No. 09/532,910, filed Mar. 22, 2000 and Ser. No. 09/390,380, filed Sep. 3, 1999. The disclosure of each of these applications is incorporated in its entirety herein by reference.

It would be advantageous to provide IOLs adapted for accommodating movement which can achieve an acceptable amount of accommodation with reduced risk of damaging the capsular bag.

### SUMMARY OF THE INVENTION

New IOLs and combinations of IOLs which provide accommodating movement in the eye have been discovered. The present IOLs are adapted and structured to take advantage of the ability of the eye to move the present IOLs sufficiently, for example, as a result of zonular tension acting on the capsular bag of the eye. The present IOLs and combinations of IOLs preferably achieve enhanced or increased accommodating movement, for example, relative to other IOLs in a similar eye. In one aspect, the present invention provides for a reduced sized optic with at least a portion or substantially all of the remaining surface of the IOL masked or non-transmitting of light passing from outside the eye toward the retina of the eye, for example, being opaque or black, to reduce spherical aberrations and to enhance optical performance. Reducing the diameter of the optic allows the radial length of the movement assembly to be increased, thereby effectively lengthening the lever arm for axial movement of the optic. This results in advantageously increasing the amount of axial movement of the optic in response to a given action of the eye. The present IOLs and IOL combinations are straightforward in construction, can be implanted or inserted into the eye using systems and procedures which are well known in the art and function effectively with little or no additional treatments or medications being required.

In one broad aspect, the present IOLs comprise an optic adapted to focus light toward a retina of an eye; and a movement assembly coupled to the optic and adapted to cooperate with the eye to effect accommodating movement of the optic. The movement assembly circumscribes the optic and comprises a member including a substantially opaque, preferably at least partially black, proximal end region coupled to the optic and a distal end region extending away from the optic and adapted to contact the capsular bag of the eye. The movement assembly circumscribing the optic very effectively enhances the degree to which the action of the eye, such as the elasticity of the capsular bag and the action of the ciliary muscle acting on the zonules and the capsular bag, causes accommodating movement of the optic. Preferably, the movement assembly is adapted to cooperate with the eye to effect accommodating movement of the optic upon radial, for example, diametrical, compression by the capsular bag of the eye.

In one very useful embodiment, the IOLs of the present invention include optics which have diameters of less than 5 mm, preferably in a range of about 2 mm to about 4 mm. Particularly useful optics in accordance with the present invention include optics having diameters in a range of about 2.5 mm to about 3.5 mm. The present IOLs preferably have optics which achieve increased accommodating movement relative to similar IOLs including optics having diameters of 5 mm. A brief simplified analysis of a disk IOL that is 10 mm in overall diameter with a 6 mm diameter optic and being angulated or vaulted so that the movement assembly is positioned at an angle of 10° with the optic being anterior of the distal end of the movement assembly is considered to assist in understanding the present invention. This IOL, under ideal conditions, for example, with no unwanted buckling or deformation, will provide a maximum of 1.02 mm of axial movement in response to 1 mm of diametrical compression of the IOL. In the present invention, with an optic having a diameter of 3 mm, and the IOL having substantially the same dimensions as noted above otherwise, the present IOL need only be compressed 0.69 mm to result in the same axial movement. Thus, the present IOLs, in particular such IOLs which have optics which have reduced diameters relative to 5 mm, achieve increased axial accommodating movement relative to similar IOLs (for example, IOLs which have the same overall diameters in the unaccommodated state) having optics with 5 mm diameters.

In a very useful embodiment of the present invention, at least a part of the proximal end region of the movement assembly is black, for example, the proximal end region comprises a black surface. Preferably, at least a portion of the distal end region of the movement assembly is substantially opaque, and more preferably at least a part of the distal end region is black, for example, the distal end region comprises a black surface. In one very useful embodiment, the entire movement assembly is substantially opaque and still more preferably is black, that is non-reflective or non-transmitting to light passing from outside the eye toward the retina of the eye. Such opaque or blackened movement assemblies reduce spherical aberrations while, at the same time, allowing useful vision correction by the reduced size optic and enhanced axial accommodating movement, as described herein.

In a very useful embodiment, the optic has a far vision correction power, more preferably a far vision correction power for infinity, in the unaccommodated state. Thus, with the IOL located in the posterior-most position, distant objects can be easily and accurately viewed.

Preferably, the movement assembly is positioned relative to the optic so that, with the IOL at rest, for example, in the eye, the optic vaults anteriorly of the distal end region of the movement assembly. This anterior vaulting feature reduces the risk of detrimental posterior stretching of the capsular bag with the IOL located in the posterior-most position in the eye. Thus, in this posterior-most position, the optic of the IOL may contact the capsular bag but, because of the anterior vaulting, causes a reduced amount of posterior stretching of the capsular bag relative to a similar IOL without the anterior vaulting feature located in the posterior-most position. The anterior vaulting feature, in addition, is effective in at least assisting in increased amounts of accommodating movement, again relative to a similar IOL without such anterior vaulting feature.

The present IOLs preferably are sized to fit the capsular bag of the eye in the unaccommodated state substantially without stretching the capsular bag. Proper sizing of the IOL facilitates enhanced accommodating movement of the IOL in the eye.

Because of the size and configuration of the present IOLs, and in particular the generally reduced diameters of the present optics, such IOLs preferably provide an amount of axial movement anteriorly in the eye in the range of about 0.6 or about 0.8 or about 2.0 mm to about 2.5 mm with about 1.0 mm of reduction in the equatorial diameter of the capsular bag. The overall diameter of the present IOLs preferably is in the range of about 8 mm to about 11 mm or about 12 mm.

The movement assembly may be adapted to be affixed to the capsular bag of the eye including the IOL.

The movement assembly preferably is sufficiently flexible to facilitate movement of the optic relative to the distal end region of the movement assembly being acted upon by the eye. The movement assembly may include a hinge assembly positioned proximally of the distal end region of the movement assembly. Such hinge assembly is effective in facilitating the accommodating movement of the optic in the eye. The hinge assembly may include one or more regions of reduced thickness, for example, circumscribing the optic. These reduced thickness regions are effective to provide a desired degree of flexibility to the movement assembly. The movement assembly may have a minimum thickness at the proximal end region and a maximum thickness at the distal end region. In one embodiment, the movement assembly includes no hole or holes passing through, for example, axially through, the movement assembly.

In a very useful embodiment, the distal end region of the movement assembly includes a peripheral edge configured to inhibit cell growth from the eye in front of or in back of the intraocular lens. In a particularly useful embodiment, the movement assembly has an anterior face and an opposing posterior face with the peripheral edge being between these two faces. The intersection of the peripheral edge and at least one of the anterior face and the posterior face forms a peripheral corner located at a discontinuity between the peripheral edge and the intersecting face. Cell growth from the eye in front of or in back of the movement assembly preferably is more inhibited relative to a substantially identical intraocular lens without the peripheral corner.

In a further broad aspect of the present invention, methods for inserting an IOL in an eye are provided. Such methods comprise providing an IOL in accordance with the present invention, as described herein. The IOL is placed into the eye, for example, in the capsular bag of the eye, using equipment and techniques which are conventional and well known in the art. The IOL is placed in the unaccommodated position in the eye. In one embodiment, the placing step is effective so that the optic of the IOL is radially, e.g., diametrically, compressed by the capsular bag, for example, by the elasticity of the capsular bag, of the eye to effect accommodating movement of the optic of the IOL. No treatments or medications, for example, to paralyze the ciliary muscle to facilitate fibrosis or otherwise influence the position of the IOL in the eye, are required. Preferably, the optic is deformed prior to being placed into the eye. Once the IOL is placed in the eye, and after a normal period of recovery from the surgical procedure, the IOL, in cooperation with the eye, provides the mammal or human wearing the IOL with near focus accommodation. In the unaccommodated state, the IOL provides the mammal or human wearing the IOL with far vision correction.

In another broad aspect of the present invention, intraocular lens combinations (ILCs) comprise a first optic, second optic and a movement assembly. The first optic, preferably having a negative optical power, is adapted to be placed in a substantially fixed position in a mammalian eye. The second optic is adapted to focus light toward a retina of an eye, and preferably has a higher optical power than the first optic. The movement assembly is coupled to the second optic and is adapted to cooperate with the eye, for example, the zonules, ciliary muscle and capsular bag of the eye, to effect accommodating movement of the second optic in the eye. The movement assembly circumscribes the second optic and comprises a member including a substantially opaque, preferably black, proximal end region coupled to the second optic and a distal end region extending away from the second optic and adapted to contact a capsular bag of the eye.

The second optics of the present ILCs preferably have the characteristics described elsewhere herein with regard to the optics of the present IOLs. For example, the second optics preferably have diameters of less than 5 mm, more preferably in a range of about 2 mm to about 4 mm and still more preferably in a range of about 2.5 mm to about 3.5 mm. Such second optics preferably achieve increased accommodating movement relative to a similar ILC including a second optic having a diameter of 5 mm.

In one useful embodiment, movement assemblies of the present ILCs are structured substantially similarly to the movement assemblies of the present IOLs, as described elsewhere herein. For example, the proximal end regions of the movement assemblies of the present ILCs preferably comprise one or more black surfaces. Also, at least a portion of, and preferably substantially all of, the distal end region of the movement assemblies of the present ILCs are substantially opaque. The benefits and advantages of the black/opaque movement assemblies of the present ILCs are substantially similar to the benefits and advantages achieved resulting from the opaque/black movement assemblies of the present IOLs.

Advantageously, the second optic has a high plus optical power to reduce the amount of movement, for example, axial movement, in the eye needed to provide accommodation for intermediate and near vision. The negative or minus optical power of the first optic compensates for the excess plus or positive optical power in the first optic. The use of such a compensating lens, that is the first optic having a negative optical power, can allow for standardization of the optical power correction in the second optic. In other words, the optical power of the second optic, that is the movable optic, can be approximately equal from optic to optic, while the optical power of the first optic, that is the fixed optic, is adjusted from optic to optic to meet the specific vision correction needs (prescription) of each individual patient. Consequently, the required amount of movement of the second optic in the eye can be approximately the same for all patients.

The present ILCs provide accommodation, preferably an acceptable degree of accommodation, in spite of movement and space limitations in the eye. For example, the maximum theoretical amount of axial movement for a simple disc lens having an overall diameter of 11 millimeters (mm) and an optic diameter of 5 mm that undergoes 1 mm of compression in its diameter is about 1.65 mm. The amount of axial movement required for a plus 15 diopter optic to provide 2.5 diopters of additional power in the spectacle plane is about 2.6 mm. However, a plus 30 diopter optic requires only 1.2 mm of axial movement to provide 2.5 diopters of additional power in the spectacle plane. Thus, by increasing the plus power of the second optic, which is adapted for accommodating movement, a reduced amount of movement is needed to achieve higher or enhanced degrees of accommodation. The first or fixed optic preferably has a minus power to compensate for the excess plus power in the second optic.

The present ILCs preferably include first and second optics with optical powers which provide a net optical power, for example, a net plus power or a net negative power, to allow light to focus on the retina. To illustrate, assume that the patient requires a plus 15 diopter correction. The first optic is provided with a minus 15 diopter optical power and the second optic with a plus 30 diopter optical power. The net optical power of this ILC is approximately the sum of minus 15 diopters and plus 30 diopters or plus 15 diopters, the desired prescription for the patient in question. The powers of the first and second optics are only approximately additive since the net power of the combination also depends on other factors including, but not limited to, the separation of the two optics, the magnitude of the power of each individual optic and its location in the eye and the like factors. Also, by adjusting the optical power of the first optic, the net optical power of the ILC can be adjusted or controlled even though the optical power of the second optic is standardized or remains the same, for example, at a plus 30 diopter optical power. By standardizing the optical power of the second optic, the amount of movement in the eye required to obtain a given level of accommodation is substantially the same, and preferably well within the space limitations in the eye, from patient to patient.

The second optic preferably is adapted to be positioned in the capsular bag of the eye.

The first optic may be coupled to a fixation member, or a plurality of fixation members, adapted to assist in fixating the first optic in the eye. Each fixation member preferably has a distal end portion extending away from the first optic. In one embodiment, the distal end portion of the fixation member is adapted to be located in the capsular bag of the eye. Alternately, the distal end portion of the fixation member may be located in contact with a sulcus of the eye. As a further alternate, the distal end portion of the fixation member may be adapted to be located in an anterior chamber of the eye.

The first optic may be located posterior in the eye relative to the second optic or anterior in the eye relative to the second optic. In a useful embodiment, the first optic is adapted to be positioned in contact with the posterior wall of the capsular bag of the eye. This positioning of the first optic provides for effective compensation of the plus or positive vision correction power of the second optic. In addition, by having the first optic in contact with the posterior wall of the capsular bag, cell growth from the capsular bag onto the ILC, and in particular onto the first and second optics of the ILC, is reduced. This, in turn, reduces the risk of or inhibits posterior capsule opacification (PCO).

In one embodiment, the fixation member or members and the movement assembly are secured together, preferably permanently secured together. Thus, when inserting the ILC into the eye, a single combined structure can be inserted. This reduces the need to position the first and second optics relative to each other. Put another way, this feature allows the surgeon to very effectively and conveniently position the ILC in the eye with reduced surgical trauma to the patient.

The fixation member and movement assembly may be secured, for example, fused, together at the distal end portion of the fixation member and the distal end region of the movement assembly.

In a useful embodiment of the present invention, the first optics have a substantially plano optical power or a negative optical power. These ILCs are particularly adapted to inhibit PCO. The first optics of these combinations preferably are adapted to be placed in a substantially fixed position in the eye. The posterior surfaces of the first optics advantageously are configured to substantially conform to a major portion, that is, at least about 50%, of the posterior wall of the capsular bag of the eye in which the combination is placed. More preferably, the posterior surfaces of the first optics are configured to substantially conform to substantially all of the posterior wall of the capsular bag. Such configuration of the first optic is very useful in inhibiting cell growth from the eye onto the first and second optics and in inhibiting PCO.

In one embodiment, the first optic has a substantially plano optical power and the second optic has a far vision correction power. In an alternate embodiment, the first optic has a negative optical power and the second optic has a positive optical power, more preferably, so that the optical powers of the first and second optics provide a net plus optical power in the eye in which the combination is placed.

In a very useful embodiment, the first optic includes an anterior surface and at least one projection extending anteriorly from this anterior surface. The at least one projection is positioned to limit the posterior movement of the second optic in the eye. Thus, the movement of the second optic is effectively controlled to substantially maintain the configuration of the combination and/or to substantially maintain an advantageous spacing between the first and second optics.

The movement assembly may be structured and functions similarly to the movement assembly of the previously described ILCs.

The first optic may have a fixation member or members coupled thereto. The fixation member or members are adapted to assist in fixating the first optic in the eye, that is in contact with the posterior wall of the capsular bag of the eye. In one embodiment, the first optic itself is configured and/or structured so that no fixation member or members are needed to maintain the first optic in contact with the posterior wall of the capsular bag of the eye. The first optic and the movement assembly of these ILCs may be secured together.

In general, the optics of the present IOLs and the first and second optics of the present ILCs may be made of any suitable materials. Preferably, these optics are made of polymeric materials. More preferably, the optics and the movement assemblies, and the fixation member(s), if any, are deformable for insertion through a small incision in the eye.

In a further broad aspect of the present invention, methods for inserting an ILC in an eye are provided. Such methods comprise providing an ILC in accordance with the present invention, as described herein. The ILC is placed into the eye, for example, in the capsular bag of the eye or partly in the capsular bag of the eye, using equipment and techniques which are conventional and well known in the art. The ILC is placed in a rest position in the eye, for example, a position so that the eye, and in particular the ciliary muscle and zonules of the eye, effectively cooperate with the movement assembly to move the second optic of the ILC anteriorly in the eye from the rest position to provide for positive accommodation. No treatments or medications, for example, to paralyze the ciliary muscle, to facilitate fibrosis or otherwise influence the position of the ILC in the eye, are required.

Preferably, the first and second optics and the movement assembly are deformed prior to being placed into the eye. Once the ILC is placed in the eye, and after a normal period of recovery from the surgical procedure, the ILC, in combination with the eye, provides the mammal or human wearing the ILC with effective accommodation, preferably with reduced risk of PCO. In the unaccommodated state, the ILC preferably provides the mammal or human wearing the ILC with far vision correction.

Any and all features described herein and combinations of such features are included within the scope of the present invention provided that the features of any such combination are not mutually inconsistent.

Additional aspects and advantages of the present invention are set forth in the following description and claims, particularly when considered in conjunction with the accompanying drawings in which like parts bear like reference numerals.

### BRIEF DESCRIPTION OF THE DRAWINGS

FIG. 1 is a fragmentary sectional view of an eye in which an IOL in accordance with the present invention has been implanted, with the lens being located in a posterior rest position in the eye.

FIG. 2 is a fragmentary sectional view of an eye in which the IOL of FIG. 3 has been implanted, with the lens being located in an intermediate position in the eye.

FIG. 3 is a fragmentary sectional view of an eye in which the IOL of FIG. 3 has been implanted with the lens being located in an anterior position in the eye.

FIG. 4 is a perspective view of the IOL shown in FIG. 1 in the rest position.

FIG. 5 is a cross-sectional view taken generally along line 5-5 of FIG. 4.

FIG. 6 is a cross-sectional view taken generally along arc 6-6 of FIG. 5.

FIG. 7 is a cross-sectional view of an another embodiment of an IOL in accordance with the present invention.

FIG. 8 is a cross-sectional view taken generally along arc 8-8 of FIG. 7.

FIG. 9 is a front plan view of an ILC in accordance with the present invention.

FIG. 10 is a cross-sectional view taken generally along line 10-10 of FIG. 9.

FIG. 11 is a cross-sectional view of an additional ILC in accordance with the present invention.

### DETAILED DESCRIPTION OF THE DRAWINGS

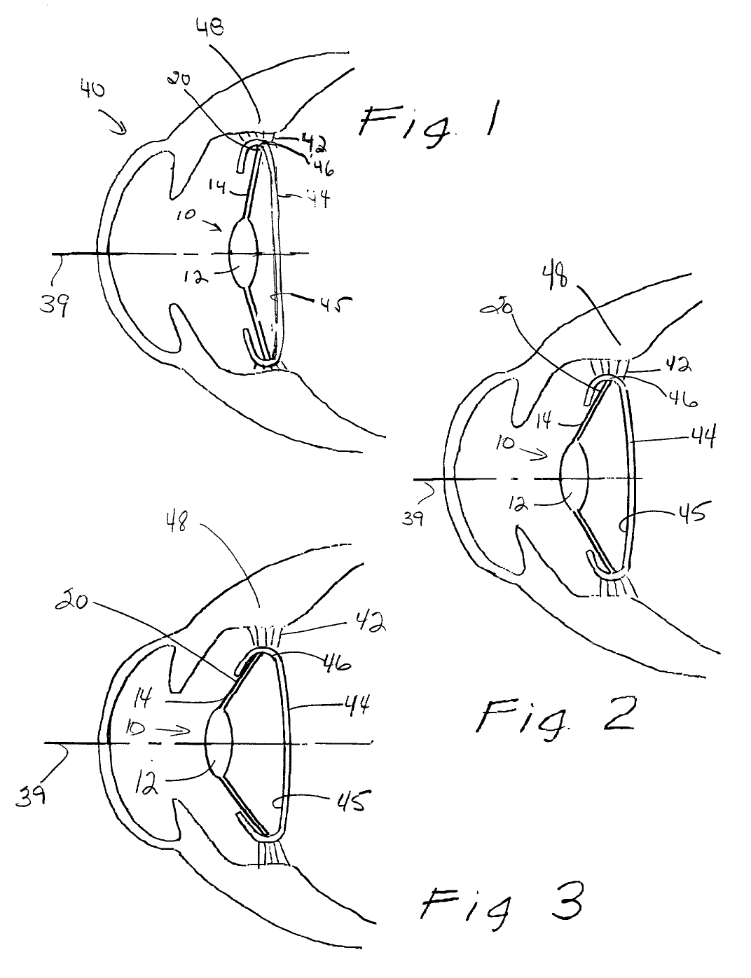

Referring now to FIGS. 1 to 5, an IOL according to the present invention, shown generally at 10, includes a lens body or optic 12 which has a diameter of 3.5 mm. Extending radially outwardly from lens body 12 is blackened member 14, which fully or completely circumscribes the lens body. Member 14, which includes no through holes, has a proximal end portion 16 which is coupled to the optic 12 at optic periphery 18. Member 14 extends radially outwardly to a distal end region 20 including a peripheral edge 22, which extends between the anterior surface 24 and the posterior surface 26 of member 14.

Member 14 extends outwardly from optic 12 sufficiently so that the distal end region 20 is in contact with the inner peripheral wall of the posterior capsular bag when the IOL 10 is implanted in the eye. As best seen in FIG. 5, when IOL 10 is at rest, the optic 12 is positioned or vaulted anteriorly relative to the distal end region 20 of member 14. In other words, the anterior surface 23 of optic 12 is anterior of the anterior surface 24 of member 14 at distal end region 20 and/or the posterior surface 25 of the optic is anterior of the posterior surface 26 of the member at the distal end region. In addition, member 14 includes an annular region 30 of relatively reduced thickness. Region 30 is effective to cause member 14 to flex relative to optic 12 in response to the action of eye 40, thereby enhancing the accommodating movement of optic 12.

The optic 12 may be constructed of rigid biocompatible materials, such as polymethyl methacrylate (PMMA), or flexible, deformable materials, such as silicone polymeric materials, acrylic polymeric materials, hydrogel polymeric materials and the like, which enable the optic 12 to be rolled or folded for insertion through a small incision into the eye. Although the optic 12 as shown is a refractive lens body, the present IOLs can include a diffractive lens body and such embodiment is included within the scope of the present invention.

Optic 12 is prescribed for the wearer of IOL 10 with a baseline or far (distance) diopter power for infinity.

The blackened member 14 may be integral (unitary) with the optic 12. Alternately, member 14 can be mechanically or otherwise physically coupled to optic 12. The member 14 fully or completely circumscribes the optic 12 to a diameter at least equal to the largest pupillary opening of the eye in which IOL 10 is to be inserted or implanted. That is, the diameter of the optic 12, shown as 3.5 mm, and the radial dimension of the blackened member 14 fully circumscribing the optic should equal at least the size of the largest pupillary opening of the eye in which the IOL 10 is to be implanted. For example, if the IOL 10 is to be implanted in a human eye, the largest pupillary opening is often in the range of about 4.5 mm or about 5.0 mm to about 6 mm or 6.5 mm. The blackened member 14, including black anterior surface 24, is effective to mask the portion of the IOL 10 other than optic 12 which may be exposed to light, from outside the eye, to reduce spherical aberrations. Of course, the entire member 14, from proximal end region 16 to distal end region 20, can be blackened, and preferably is for ease of manufacture and enhanced masking benefits. Although the member 14, outside the radial dimension which may be exposed to light from outside the eye, may include holes and/or may not fully circumscribe the optic and/or may not be blackened, it is preferred that the entire member 14 be solid and/or fully circumscribe the optic and/or be blackened.

The member 14 may be constructed of the same or different biocompatible materials as optic 12, and preferably is made of polymeric materials, such as polypropylene, silicone polymeric materials, acrylic polymeric materials and the like. Member 14 is blackened so as to be substantially non-reflective or non-transmitting to light to which the blackened member is exposed. Such blackening can be provided for by any suitable method and/or means. As shown in FIG. 1 to 5, the entire member 14 is blackened, for example, by including carbon particles or black dye or pigment in the polymeric material used to produce the member. Alternately, the anterior surface 24 of member 14 can be painted or otherwise coated with a black coating material to provide the desired blackening. In any event, the member 14, particularly from the proximal end region 16 extending radially outwardly a distance beyond which the member 14 is exposed to light from outside the eye, is sufficiently opaque or non-transmitting to light so that the portion of the member, or the entire member, does not transmit any substantial amount of light to the retina of the eye in which the IOL 10 is implanted.

The methodology by which the member 14 is made non-transmitting to light should, of course, have no significant or undue adverse effect on the structure and functioning of the member. In addition, the material, if any, remaining with the member 14 to render it non-transmitting should be biocompatible or ophthalmically acceptable in or on the member in the eye. Preferably, any such material or materials remain secured to the member 14 on a long term basis after the IOL 10 is placed in the eye.

Member 14 has sufficient strength or rigidity to be effective to transfer the force from the capsular bag of the eye to move the optic 12 axially in the eye to effect accommodation. Such strength or rigidity is enhanced by employing a solid member 14, that is a member having no axial through hole or holes, for example, perforations. The member 14 preferably is deformable, in much the same manner as optic 12 is deformable, to facilitate passing IOL 10 through a small incision into the eye. The material or materials of construction from which member 14 is made are chosen to provide the member with the desired mechanical properties, e.g., strength, and/or deformability, to meet the needs of the particular application involved.

The IOL 10 can be made in any suitable manner, many of which are well known in the art. For example, insert molding can be employed to provide IOL 10 with optically clear optic 12 and blackened member 14. Machining, e.g., lathing and the like, an/or other conventional or well known methodologies may also be employed.

The IOL 10 can be inserted into the capsular bag of a mammalian eye using conventional equipment and techniques, for example, after the natural crystalline lens of the eye is removed, using a phacoemulsification technique. The IOL 10 preferably is rolled or folded prior to insertion into the eye, and is inserted through a small incision, on the order of about 3.2 mm, into the eye and is located in the eye 40, as shown in FIGS. 1 to 3.

The IOL 10 in the eye 40, as shown in FIG. 1, with the zonules 42 under tension is located in a posterior position in the capsular bag 44. The configuration of IOL 10, in particular with regard to the anterior vaulting of the optic 12, allows the IOL to be in the posterior-most position in the eye with the optic in close proximity to or even contacting the posterior inner wall 45 of the capsular bag 44. However, in the posterior-most position the IOL 10 does not cause substantial stretching of the capsular bag 44. The natural elasticity of the capsular bag preferably is substantially maintained and is effective in providing accommodating movement of the IOL 10.

The IOL 10 is positioned so that the optic 12, in cooperation with the eye 40, can be moved axially, substantially along optical axis 39 in the eye to provide accommodation.

The distal end region 20 of member 14 is in contact with the interior peripheral wall 46 of the capsular bag 44. Over time, the distal end region 20 of the member 14 may become affixed to the capsular bag 44, although this is not necessary to obtain benefits in accordance with the present invention. The member 14, in the eye 40, cooperates with the eye to effect accommodating movement of the optic 12, preferably upon radial, such as diametrical, compression of the IOL 10 by the elastic capsular bag 44 of the eye.

The IOL 10 is sized to facilitate the movement of the optic 12 in response to the action of ciliary muscle 48 and zonules 42. For example, the optic 12 is sized relatively small, that is 3.5 millimeters in diameter, to facilitate providing an increased amount of accommodating movement. The reduced size of optic 12, is effective to focus light on the retina of eye 40 and together with blackened member 14 allow, not only an increased amount of accommodating movement, but also reduced spherical aberrations and reduced glare. If the IOL 10 is to be included in an adult human eye, the optic 12 preferably has a diameter of less than bout 5 mm, more preferably in the range of about 2 mm to about 4 mm, more preferably in the range of about 2.5 mm to about 3.5 mm or about 4 mm and the IOL has an overall maximum diameter, with the member 14 in the unflexed or at rest state, in the range of about 8 mm to about 11 mm or about 12 mm.

The zonules 42 and the ciliary muscle 48 are effective to reduce or increase the equatorial diameter of the capsular bag 44 and thereby move the IOL 10 included in the bag anteriorly or posteriorly, respectively. Thus, relaxation of the ciliary muscle 46 causes the zonules 42 to increase the equatorial diameter of the capsular bag 44, resulting in IOL 10 moving posteriorly into a posterior position, as shown in FIG. 1. The reduced size of the optic 12 results in the member 14 having an enlarged radial dimension. Thus, the optic 12 is coupled to the capsular bag 44 by a longer lever arm which, in response to the action of the eye 40, increases the amount of accommodating movement achievable by optic 12. The anterior vault or angulation of optic 12 relative to member 14 further enhances the amount of accommodating movement that optic 12 is provided with.

With IOL 10 in the posterior position, as shown in FIG. 1, far away or distant objects are brought into focus.

If a near object is to be viewed, the ciliary muscle 48 contracts or constricts causing a reduction in the tension of the zonules 42, which allows the equatorial diameter of the capsular bag 44 to reduce. The IOL 10 is thereby diametrically compressed and moved anteriorly, as shown in FIG. 3. Without wishing to limit the invention to any particular theory of operation, it is believed that the capsular bag 44 has or retains sufficient elasticity to act directly on the IOL 10 to compress the IOL 10 and move the IOL 10 anteriorly. This action of ciliary muscle 48, zonules 42 and capsular bag 44 causes member 14 to flex or vault into an anterior position, shown in FIG. 3, which further enhances or increases (amplifies) the amount of anterior movement of optic 12. This anterior vaulting action of member 14, together with the anterior vaulting of optic 12 and the reduced size of optic 12 and the reduced size of optic 12 (increased lever arm length), increases the amount of positive (near) accommodating movement of optic 12 relative to a similar IOL having an optic with a diameter of 5mm. In effect, IOL 10 achieves increased accommodating movement because of a reduced size optic and such vaulting. This anterior movement of optic 12 provides near focus accommodation to allow the near object to be viewed.

The present IOL 10 has the ability, in cooperation with the eye, to move both posteriorly and anteriorly in the eye, to provide for both distance focus and near focus, respectively. This movement of IOL 10 advantageously occurs in response to action of the ciliary muscle 48, zonules 42 and capsular bag 44 which action is substantially similar to that which effects accommodation in an eye having a natural crystalline lens. Thus, the ciliary muscle 48, zonules 42 and capsular bag 44 require little, if any, retraining to function in accordance with the present invention. The member 14, as described herein, preferably is effective to facilitate or even enhance or accentuate the axial movement of the IOL 10 caused by the action of the ciliary muscle 48, zonules 44 and capsular bag 44 to provide increased degree of accommodation.

IOL 10 is such that the amount of positive or near accommodation preferably is in the range of about 1 to about 2.5 or about 3.5 diopters or more. Looked at from another perspective, the configuration and sizing of IOL 10 is effective to provide an amount of axial movement anteriorly in the eye in a range of about 0.6 mm or about 0.8 mm or about 2.0 mm to about 2.5 mm with about 1 mm of reduction in the equatorial diameter of the capsular bag 44 caused by the action of the ciliary muscle 48 and zonules 42. This amount of axial movement is based on an initial position of the IOL 10 in the posterior position, as shown in FIG. 1.

As best shown in FIG. 6, the intersections of peripheral edge 22 with the anterior face 24 and posterior face 26 of member 14 also are at substantially 90° relative to the optical axis of the IOL 10. These sharp corners 41 and 43, which involve substantial discontinuities, rather than continuous or curved transitions, between the peripheral edge 22 and anterior face 24 and posterior face 26, respectively, have been found to be effective in inhibiting or retarding cell migration or growth from the eye onto or over the optic 12 of the IOL 10.

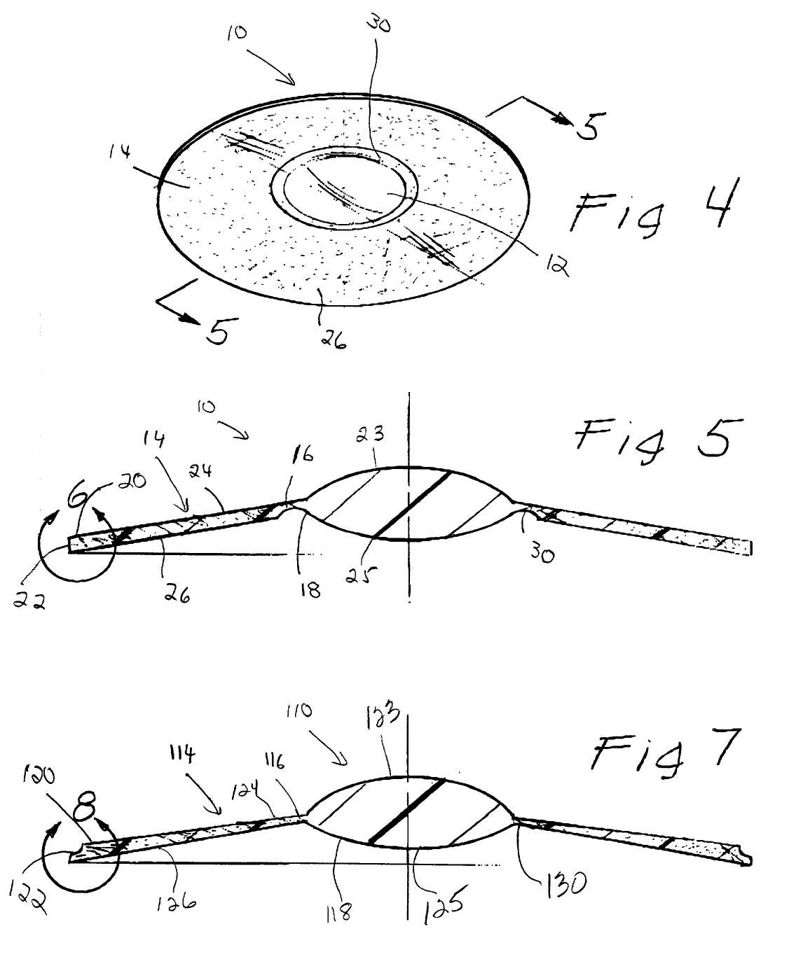

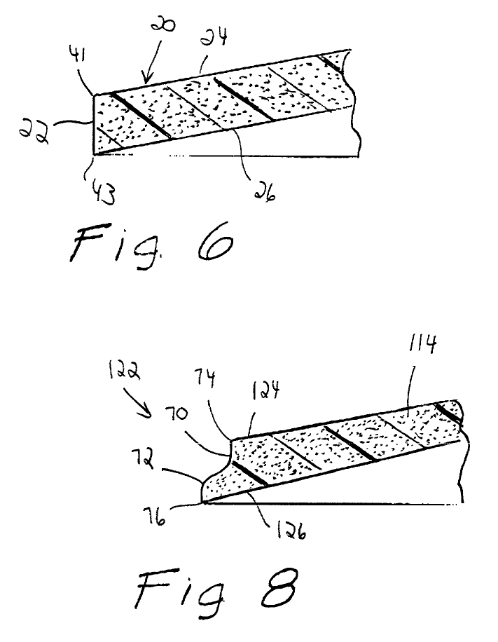

FIGS. 7 and 8 illustrate an additional IOL, shown generally at 110, in accordance with the present invention. Except as expressly described herein, additional IOL 110 is structured and functions similarly to IOL 10. Components of IOL 110 which correspond to components of IOL 10 are indicated by the same reference numeral increased by 100.

One primary difference between IOL 110 and IOL 10 relates to the configuration of member 114. In particular, as best shown in FIG. 6, member 114 is configured in a tapered manner so that the proximal end region 116 has a minimum thickness and distal end region 120 has a maximum thickness. This tapered configuration of member 114 is effective in a manner similar to region 30 of IOL 10 to cause flexing of the IOL 110, particularly with the equatorial diameter of the capsular bag being reduced. This tapered configuration of member 114 can be considered substantially equivalent to the member 14 including the reduced thickness region 30. Both of these configurations can be looked at as including a hinge located in proximity to the proximal end regions 16 and 116 of members 14 and 114, respectively.

An additional difference between IOL 110 and IOL 10 has to do with the configuration of peripheral edge 122.

With specific reference to FIG. 8, peripheral edge 122 includes a first portion 70 which is concave relative to the optical axis of IOL 110. Peripheral 122 also includes a second portion 72 which is convex relative to the optical axis of IOL 110. Thus, the curvature of the peripheral edges of the present IOLs, for example, peripheral edge 122 of IOL 110, can be relatively complex. In addition, the peripheral edge 122 intersects anterior face 124 of member 114 at peripheral corner 74 at an angle of about 90°. Similarly, peripheral edge 122 intersects the posterior face 126 of member 114 at posterior peripheral corner 76 at an angle of about 90°. The peripheral anterior corner 74 and peripheral posterior corner 76 are effective in inhibiting or retarding cell migration or growth from the eye onto or over the optic 112.

Other peripheral edge configurations may be employed to inhibit or retard the migration of cells from the eye onto the optic of the IOL. For example, the peripheral edge can include a champhered portion intersecting the anterior face of the member, preferably at a discontinuity, an intermediate portion extending outwardly and posteriorly from the champhered portion at an angle other than parallel to the central optical axis of the optic, and a flat or posterior portion extending from the intermediate portion and intersecting the posterior face of the member, preferably at a discontinuity. This flat portion advantageously is parallel to the central optical axis of the optic.

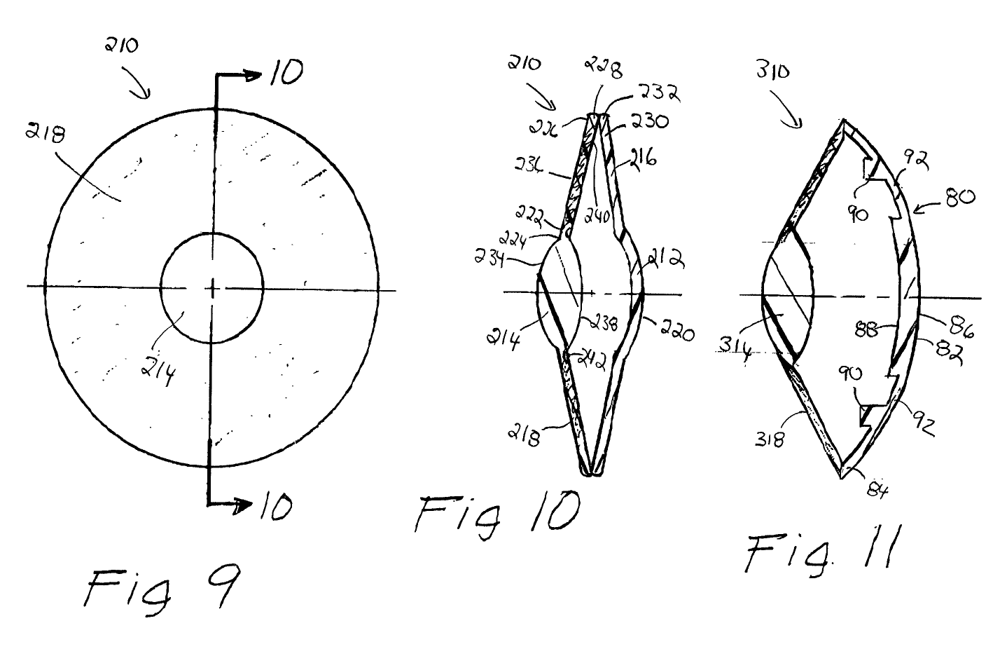

Referring now to FIGS. 9 and 10, an ILC according to the present invention, shown generally at 210, includes a first optic 212, a second optic 214, a disc type fixation member 216 and a disc type movement assembly 218.

The first optic 212 has substantially plano optical power and is adapted to be held in a fixed position, for example, at least partially by the fixation member 216. When the ILC 210 is positioned in a human eye, the posterior surface 220 of first optic 212 is in contact with the inner posterior wall of the capsular bag of the eye. This positioning of optic 212 is very effective in reducing or inhibiting endothelial cell growth from the capsular bag onto the first optic 212. In effect, the positioning of the first optic 212 against the posterior surface of the capsular bag inhibits or reduce the risk of

The second optic 214 includes a distance vision correction power. Except as expressly described herein, second optic 214 is sized, structured and functions similarly to optic 12 of IOL 10. The movement assembly 218 extends radially outwardly from second optic 214 and fully circumscribes the second optic 214. Movement assembly 218 has a proximal end region 222 which is coupled to the second optic 214 at first optic periphery 224.

Movement assembly 218 extends radially outwardly to a distal end region 226 including a peripheral zone 228. Except as expressly described herein, movement assembly 218 is sized, structured and functions similarly to member 14 of IOL 10.

Fixation member 216 includes a distal end portion 230 including a peripheral area 232. The movement assembly 218 and fixation member 216 are fused together at the peripheral zone 228 and peripheral area 232. Thus, the entire ILC 210 is a single unitary structure. The first optic 212 and fixation member 216 can be manufactured separately from second optic 214 and movement assembly 218 and, after such separate manufacture, the fixation member and movement assembly can be fused together. Alternately, the entire ILC 210 can be manufactured together. Also, if desired, the first optic 212 and fixation member 216 can be inserted into the eye separately from the second optic 214 and movement assembly 218. Thus, ILC 210 can comprise a plurality of separate components.

Movement assembly 218 extends outwardly from second optic 214 sufficiently so that the distal end region 226, and in particular the peripheral zone 228 of the distal end region, is in contact with the inner peripheral wall of the posterior capsular bag when the ILC 210 is implanted in the eye.

As best seen in FIG. 10, when ILC 210 is at rest, the second optic 214 is positioned vaulted anteriorly relative to the distal end region 226 of movement assembly 218. In other words, the anterior surface 234 of second optic 214 is anterior of the anterior surface 236 of movement assembly 218 at distal end region 226 and/or the posterior surface 238 of the second optic 214 is anterior of the posterior surface 240 of the movement assembly at the distal end region.

The first optic 212 may be constructed of rigid biocompatible materials, such as polymethyl methacrylate (PMMA), or flexible, deformable materials, such as silicone polymeric materials, acrylic polymeric materials, hydrogel polymeric materials, and the like, which enable the optic 212 to be rolled or folded for insertion through a small incision into the eye. In one embodiment, the diameter of the first optic 212 is greater than the diameter of the second optic 214. Although the first and second optics 212 and 214 as shown are refractive lens bodies, the present ILCs can include at least one diffractive lens body, and such embodiment is included within the scope of the present invention.

As noted previously, first optic 212 has a substantially plano or zero optical power. Second optic 214 is prescribed for the wearer of ILC 210 with a baseline or far (distance) diopter power for infinity. Thus, the wearer of ILC 210 is provided with the vision correction power of second optic 214 with little or no contribution from the first optic 212.

The fixation member 216 as shown, is integral (unitary) with and circumscribes the first optic 212. Alternately, fixation member 216 can be mechanically or otherwise physically coupled to first optic 212. Also, the fixation member 216 may only partially circumscribe first optic 212, and such embodiment is included within the scope of the present invention. The fixation member 216 may be constructed from the same or different biocompatible materials as first optic 212, and preferably is made of polymeric materials, such as polypropylene silicone polymeric materials, acrylic polymeric materials, and the like.

Movement assembly 218 includes a region of reduced thickness 242 located at the proximal end region 222. This area of reduced thickness, which completely circumscribes the second optic 214, acts as a hinge to provide additional flexibility to the movement member 218 to extenuate or amplify the accommodating movement of second optic 214 in response to the action of the ciliary muscle and zonules.

The fixation member 216 and movement assembly 218 preferably are deformable, in much the same manner as first and second optics 212 and 214 are deformable, to facilitate passing ILC 210 through a small incision into the eye. The material or materials of construction from which fixation member 216 is made are chosen to provide such member with the desired mechanical properties, e.g., strength and/or deformability, to meet the needs of the particular application involved.

The ILC 210 can be inserted into the capsular bag of a mammalian eye using conventional equipment and techniques, for example, after the natural crystalline lens of the eye is removed, such as by using a phacoemulsification technique. The ILC 210 preferably is rolled or folded prior to insertion into the eye, and is inserted through a small incision into the eye and is located in the capsular bag of the eye.

The ILC 210 in the eye is located in a position in the capsular bag so that the posterior surface 220 of first optic 212 is maintained in contact with the inner posterior wall of the capsular bag. As noted previously, positioning the first optic 212 in contact with the posterior wall of the capsular bag reduces the risk of or inhibits cell growth from the capsular bag onto the first optic 212 which, in turn, reduces or inhibits PCO. The ciliary muscle and zonules of the eye provide force sufficient to move axially second optic 214 sufficiently to provide accommodation to the wearer of ILC 210.

The ILC 210 should be sized to facilitate the movement of the second optic 214 in response to the action of the ciliary muscle and zonules of the eye in which the ILC is placed.

If the ILC 210 is to be included in an adult human eye, the first optic 212 preferably has a diameter in the range of about 3.5 mm to about 7 mm, more preferably in the range of about 5 mm to about 6 mm. The ILC 210 preferably has an overall maximum diameter, with the fixation member 216 and movement member 218 in the unflexed or rest state, in the range of about 8 mm to about 11 mm or about 12 mm.

The present ILC 210 has the ability, in cooperation with the eye, to move the second optic 214 both posteriorly and anteriorly in the eye, to provide for both distance focus and near focus, respectively. This movement of ILC 210 advantageously occurs in response to action of the ciliary muscle and zonules, which action is substantially similar to that which effects accommodation in an eye having a natural crystalline lens. Thus, the ciliary muscle and zonules require little, if any, retraining to function in accordance with the present invention. The movement member 218, as described herein, preferably is effective to facilitate or even enhance or extenuate the axial movement of the second optic 214 caused by the action of the ciliary muscle and zonules to provide increased degree of accommodation.

FIG. 11 illustrates an additional ILC, shown generally at 310, in accordance with the present invention. Except as expressly described herein, ILC 310 is structured and functions similar to ILC 210. Components of ILC 310 which correspond to components of ILC 210 are indicated by the same reference numeral increased by 100.

One primary difference between ILC 310 and ILC 210 relates to the substitution of a posterior lens structure 80 for the first optic 212 and fixation member 216. Lens structure 80 includes a posterior face 82 which is configured to come in contact with and substantially conform to the inner posterior surface of the capsular bag of the eye in which the ILC 310 is to be placed. Thus, the surface 82 which extends around the peripheral area 84 and across the center region 86 of the lens structure 80 is adapted to come in contact with and substantially conform to the inner posterior wall of the capsular bag. Moreover, the lens structure 80 is adapted to remain in contact with this inner posterior wall of the capsular bag and to be fixed in the eye. This configuration has been found to be very effective in inhibiting cell growth from the eye onto the ILC 310. The anterior surface 88 of lens structure 80 is configured to provide the lens structure with a substantially plano or zero optical power. Second optic 314 is prescribed for the wearer of ILC 310 with a baseline or distance or far (distance) dioptic power for infinity. Thus, the wearer of ILC 310 is provided with a vision correction power of second optic 314 with little or no contribution from the lens structure 80.

Alternately, second optic 314 has a high plus power, for example, plus 30 diopters. The lens structure 80, and in particular the region of the lens structure, defined by the anterior surface 88, which extends substantially across the entire field of vision of the wearer of ILC 310, has a minus vision correction power which is controlled to provide the correction prescription for use in the eye in which the ILC 310 is placed. For example, if this eye requires a plus 15 diopter power, the lens structure 80 has a vision correction power of approximately minus 15 diopters so that the net vision correction power of the combination of lens structure 80 and second optic 314, is plus 15 diopters.

The lens structure 80 can be made from materials described previously with regard to first optic 212 and fixation member 216.

One additional feature of lens structure 80 relates to the anteriorly extending projections 90 which extend from the base element 92 of lens structure 80. The number of these projections 90 can range from 2 to about 6 or more. Alternately, a continuous annulus projecting anteriorly can be provided. The purpose of the projections 90 or the continuous annulus is to limit the posterior movement of the second optic 314 and movement assembly 318. This limitation in the movement provides an additional degree of control of the ILC 310, and prevent a collapse of the ILC 310 and maintains an advantageous degree of separation between second optic 314 and anterior surface 88 of lens structure 80.

The present invention provides accommodating IOLs, ILCs and methods for obtaining accommodation using such IOLs and ILCs. The present IOLs and ILCs are configured to obtain increased amounts of accommodation to reduce the stretching of the capsular bag, to maintain the elasticity and/or integrity of the capsular bag, to enhance the effectiveness of the eye in providing accommodating movement of the IOL or ILC in the eye and to inhibit or retard cell growth from the eye onto the object of the IOL. These benefits are obtained with IOLs and ILCs which are straightforward in construction, relatively easy to manufacture and insert into the eye and which are effective to provide accommodation for long term use.

While this invention has been described with respect to various specific examples and embodiments, it is to be understood that the invention is not limited thereto and that it can be variously practiced within the scope of the following claims.

What is claimed is:

1. An intraocular lens comprising:
an optic adapted to focus light toward a retina of an eye; and
a movement assembly coupled to the optic and adapted to cooperate with the eye to effect accommodating movement of the optic in the eye, the movement assembly circumscribes the optic and comprises a member including a substantially opaque proximal end region coupled to the optic and a distal end region extending away from the optic and adapted to contact a capsular bag of the eye.

2. The intraocular lens of claim 1 wherein the optic has a diameter of less than 4 millimeters.

3. The intraocular lens of claim 1 herein the optic achieves increased accommodating movement relative to a similar intraocular lens including an optic having a diameter of 5 millimeters.

4. The intraocular lens of claim 1 wherein the movement assembly is positioned relative to the optic so that, with the intraocular lens at rest, the optic vaults anteriorly of the distal end region of the movement assembly.

5. The intraocular lens of claim 1 wherein at least a part of the proximal end region is black.

6. The intraocular lens of claim 1 wherein at least a portion of the distal end region is substantially opaque.

7. The intraocular lens of claim 1 wherein the optic has a far vision correction power for infinity in the unaccommodated state.

8. The intraocular lens of claim 1 wherein the distal end region of the movement assembly includes a peripheral edge configured to inhibit cell growth from the eye in front of or in back of the intraocular lens.

9. The intraocular lens of claim 1 sized to provide an amount of axial movement anteriorly in the eye in a range of about 0.6 mm to about 2.5 mm with about 1 mm of reduction in an equatorial diameter of the capsular bag.

10. The intraocular lens of claim 1 wherein the optic has a diameter in a range of about 2 millimeters to about 4 millimeters.

11. The intraocular lens of claim 1 which is deformable for insertion through a small incision in the eye.

12. The intraocular lens of claim 1 wherein the movement assembly is sufficiently flexible to facilitate movement of the optic relative to its distal end region upon being acted upon by the eye.

13. The intraocular lens of claim 1 wherein the movement assembly has a minimum thickness at the proximal end region and a maximum thickness at the distal end region.

14. The intraocular lens of claim 1 wherein the movement assembly includes a hinge positioned proximally of the distal end region.

15. The intraocular lens of claim 14 wherein the hinge includes a region of reduced thickness circumscribing the optic.

16. A method for inserting an intraocular lens in an eye, the method comprising:
providing an intraocular lens of claim 1; and
placing the intraocular lens in the capsular bag of the eye so that the eye effectively cooperates with the intraocular lens to move the optic of the intraocular lens anteriorly in the eye to provide for positive focus accommodation.

17. The method of claim 16 wherein the placing step is effective so that the intraocular lens is radially compressed by the capsular bag of the eye to effect accommodating movement of the optic of the intraocular lens.

18. An intraocular lens combination comprising:
a first optic adapted to be placed in a substantially fixed position in an eye;
a second optic adapted to focus light toward a retina of an eye; and
a movement assembly coupled to the second optic and adapted to cooperate with the eye to effect accommodating movement of the second optic in the eye, the movement assembly circumscribes the second optic and comprises a member including a substantially opaque proximal end region coupled to the second optic and a distal end region extending away from the second optic and adapted to contact a capsular bag of the eye.

19. The combination of claim 18 wherein the second optic has a diameter of less than 5 millimeters.

20. The combination of claim 18, wherein the second optic otherwise increased accommodating movement relative to a similar combination including a second optic having a diameter of 5 millimeters.

21. The combination of claim 18 wherein the second optic has a diameter in a range of about 2 millimeters to about 4 millimeters.

22. The combination of claim 1 wherein at least a part of the proximal end region is black.

23. The combination of claim 18 wherein at least a portion of the distal end region is substantially opaque.

24. The combination of claim 18 wherein the second optic is adapted to be positioned in a capsular bag of the eye.

25. The combination of claim 24 wherein the first optic is coupled to a fixation member adapted to assist in fixating the first optic in the eye and having a distal end portion extending away from the first optic.

26. The combination of claim 25 wherein the distal end portion is adapted to be located in the capsular bag of the eye.

27. The combination of claim 18 wherein the first optic is adapted to be located posterior in the eye relative to the second optic.

28. The combination of claim 27 wherein the first optic is adapted to be positioned in contact with a posterior wall of a capsular bag of the eye.

29. The combination of claim 18, wherein the optical power of the first optic and the optical power of the second optic are controlled to provide vision correction and accommodation in the eye in which the optics are located.

30. The combination of claim 18 wherein a first optic has a posterior surface adapted to be positioned in contact with a posterior wall of a capsular bag of an eye.

31. The combination of claim 30 wherein the first and second optics and the movement assembly are deformable for insertion through a small incision in the eye.

32. The combination of claim 30 wherein the movement assembly is sufficiently flexible to facilitate movement of the second optic in the eye upon being acted upon by the eye.

33. The combination of claim 30 wherein the movement assembly includes a hinge.
